(19) United States
(12) Patent Application Publication
(10) Pub. No.: US 2017/0000727 A1
(43) Pub. Date: Jan. 5, 2017

(54) DRY ENEMA PRODUCT

(71) Applicant: DISPHAR INTERNATIONAL B.V., Hengelo (NL)

(72) Inventors: José Luis VELADA, Amersfoort (NL); Hiteshkumar Anilkant DOSHI, Mumbai (IN); Shruti Ashok HAZARE, Mumbai (IN)

(73) Assignee: DISPHAR INTERNATIONAL B.V., Hengelo (NL)

(21) Appl. No.: 15/104,284

(22) PCT Filed: Dec. 18, 2014
(86) PCT No.: PCT/NL2014/050881

§ 371 Date: Jun. 14, 2016

(30) Foreign Application Priority Data
Dec. 20, 2013 (EP) 13199035.0

(51) Int. Cl. A61K 9/00; A61K 9/16; A61K 31/606

(52) U.S. Cl. CPC A61K 9/0031; A61K 31/606; A61K 9/1611; A61K 9/1652

(57) ABSTRACT

The invention relates to a pharmaceutical dry enema product. Said product comprises mesalazine dry granules suitable for reconstitution into enema. The pharmaceutical product preferably is supplied in a package together with an enema bottle with one-way valve. The product can be easily dispersed and after reconstitution it has required viscosity to ensure uniform suspension of the drug and better retention in inflamed mucosa and it can be applied without loss or spillage of the pharmaceutical active ingredient.

### FIELD OF THE INVENTION

The present invention relates to a new dry enema product comprising a granulate with mesalazine as an active ingredient together with an enema bottle having a one-way valve.

### BACKGROUND OF THE INVENTION

Mesalazine (5-aminosalicylic acid) is known for its anti-inflammatory properties and has been found effective in the treatment of inflammatory bowel disease (IBD) such as ulcerative colitis as well as in the treatment of mild to moderate Crohn's disease. Ulcerative colitis, also referred to as UC, is the most common inflammatory bowel disease and affects various portions of the gastrointestinal tract (GI), particularly the lower GI tract, and more particularly the colon and/or rectum. Crohn's disease predominates in the small and the large intestine. Diseased patients usually have deeper inflammations in the most distal part of the small intestine and the first part of the large intestine (ileocaecal region), but the inflammation can be located in any part of the gastrointestinal tract. Current treatments focus both on oral and rectal administration of the mesalazine.

Commercially available rectal forms are ready-to-use suspension enemas that are, functionally, retention enemas. The suspensions usually require use of preservatives so as to increase their shelf life. Many of such preservatives, being irritants to colon mucosa, are to be preferably avoided in such rectally deliverable suspensions. Further, in view of volume and weight considerations, liquid formulations are uneconomical to transport and hence dry mesalazine granulates for reconstitution into enema are preferred. No such dry enema product is commercially available yet.

There have been attempts in the past to prepare granules of mesalazine that can readily disintegrate to form suspensions. U.S. Pat. No. 6,261,602 discloses granules that can disintegrate in water to provide a suspension of mesalazine that can be delivered orally. However, such suspensions for oral intake do not possess the specific viscosity which is required for a retention enema. Additionally, such suspensions for oral intake do not have the desired isotonicity, required for retention enemas.

US 2012/0149667 describes a granulate comprising a combination of both mesalazine and prednisolone as active, which may suitably be used for reconstitution into an enema. This document specifically teaches the advantageous use of the combination of mesalazine and prednisolone and does not disclose any details about viscosity.

### SUMMARY OF THE INVENTION

Hence, although the prior art discloses several compositions directed to specific isolated sub-parts of galenical development of mesalazine granulates, there is a need to provide value-added products with improved patient use, wherein multiple improvements are combined into a commercially attractive product which overcomes the problems as indicated above.

According to a first aspect, the invention is directed to a pharmaceutical composition comprising mesalazine granules suitable for reconstitution into an enema and an enema bottle comprising a one-way valve.

According to one embodiment, the pharmaceutical composition of the present invention comprises an enema bottle which is bio-degradable.

In other words, the invention concerns a kit of parts comprising mesalazine granules suitable for reconstitution into an enema and an enema bottle comprising a one-way valve. In a preferred embodiment, the enema bottle of the kit of parts is bio-degradable.

In yet another embodiment, the mesalazine granules in the pharmaceutical composition or the kit of parts of the present invention comprise an isotonicity agent, a viscosity agent and/or a disintegrating agent.

In another aspect, the invention concerns a dry enema preparation comprising mesalazine granules that comprise an isotonicity agent, a viscosity agent and/or a disintegrating agent. In a preferred embodiment, the dry enema preparation comprises mesalazine granules that comprise an isotonicity agent, a viscosity agent and a disintegrating agent.

According to another embodiment of the pharmaceutical composition or of the kit of parts or of the dry enema preparation according to the invention, the isotonicity agent and the disintegrating agent are present in a ratio by weight ranging from 1:0.8 to 1:2.5, preferably 1:0.8 to 1:1.5, more preferably in a ratio by weight ranging from 1:0.9 to 1:1.2.

According to another embodiment of the pharmaceutical composition or of the kit of parts or of the dry enema preparation according to the invention, the disintegrating agent and the viscosity agent are present in a ratio by weight ranging from 1:0.1 to 1:0.5, preferably in a ratio by weight ranging from 1:02 to 1:0.5.

According to another embodiment, the mesalazine granules comprise, based on the total weight of the granules, from 30% to 70% w/w of mesalazine, preferably from 34 to 65% w/w; and from 5% to 15% w/w of a viscosity agent, preferably from 7% to 13% w/w; and from 10% to 30% w/w of a disintegrating agent, preferably from 15% to 25% w/w; and from 10% to 30% w/w of an isotonicity agent, preferably from 13% to 28% w/w.

According to another embodiment of the pharmaceutical composition or of the kit of parts or of the dry enema preparation according to the invention, the isotonicity agent that is included comprises sodium chloride and preferably the isotonicity agent is sodium chloride.

According to another embodiment of the pharmaceutical composition or of the kit of parts or of the dry enema preparation according to the invention the viscosity agent that is included comprises a gum, and preferably the viscosity agent is a gum.

According to another embodiment of the pharmaceutical composition or of the kit of parts or of the dry enema preparation according to the invention, the disintegrating agent comprises croscarmellose sodium and preferably the disintegrating agent is croscarmellose sodium.

According to another embodiment of the pharmaceutical composition or of the kit of parts or of the dry enema preparation according to the invention, the mesalazine granules when reconstituted with water to a volume of 100 ml at 25° C. forms a suspension having a viscosity greater than 200 cps (centipoise; 1 cps=1 mPa·s) and less than 500 cps, preferably ranging from 220 cps to 480 cps.

Viscosity is measured at 25° C. using a Brookfield's viscometer equipped with spindle 3 operated at 50 rpm.

According to another embodiment, the pharmaceutical composition or kit of parts or the dry enema preparation of the present invention comprise mesalazine or its pharmaceutically acceptable salt in an amount of 500 to 5000 mg.

According to another embodiment, the mesalazine granules are present in a monodose container such as a sachet or a stick pack.

### DETAILED DESCRIPTION OF THE INVENTION

It is an object of the present invention to provide a novel mesalazine dry granulate composition for rectal administration which can easily be dispersed and which after reconstitution has required viscosity to ensure uniform suspension of the drug and theoretically better retention in inflamed mucosa. At the same time, the composition should be easy to prepare and easy to apply without loss or spillage of the drug. Hence the present invention provides a pharmaceutical composition or kit of parts comprising mesalazine granules suitable for reconstitution into an enema and an enema bottle comprising a one-way valve.

In yet another aspect of the present invention, mesalazine granules are provided which comprise an isotonicity agent. Isotonicity agents can be added to enema preparations to reduce local irritation by preventing osmotic shock at the site of application. For comfort during administration, the enema dosage form is preferably “isotonic” with body fluids. The use of isotonicity agents in general results in a drop of viscosity of the suspension, and hence have a negative effect on the formation of retention enemas.

The isotonicity agent that is used in the granules of the invention preferably is an electrolyte or a non-electrolyte that is not an irritant to the colon mucosa and that can bring the tonicity of the suspension to match the tonicity of the body fluid. Preferably, the tonicity agent is selected from the group consisting of sodium chloride, dextrose, mannitol and sorbitol. More preferably, sodium chloride is used as the isotonicity agent. The isotonicity agent preferably is present in an amount from 10% to 30% w/w, preferably from 13% to 28% w/w, based on the total weight of the granules.

In one embodiment of the present invention, the mesalazine granules contain a viscosity agent. Such viscosity agent suitably provides sufficient viscosity to the reconstituted suspension in order to prevent it from forming a sediment on the bottom of the enema bottle. At the same time, said suspension preferably also has a viscosity which is sufficient to retain the suspension after its application into the rectal cavity but on the other hand the viscosity preferably is not too high since this would make the rectal delivery unnecessary difficult. Viscosity agents, that usually possess binding characteristics, normally tend to delay the disintegration of the granules. The viscosity agents that can be used to prepare the granules of the invention include, but are not limited to, gums such as xanthan gum, guar gum, acacia gum, or cellulose derivatives such as hydroxypropyl methyl cellulose (HPMC), methyl cellulose, or carbomers, polyvinyl alcohol, pectin, alginic acid and salts thereof. Preferably, xanthan gum is used as the viscosity agent in preparing the granules of the invention. The viscosity agent preferably is present in an amount from 5% to 15% w/w, preferably it from 7% to 13% w/w, based on the total weight of the granules.

In one embodiment of the present invention, the mesalazine granules comprise a disintegrating agent. Contrary to the popular understanding that disintegrating agents swell without contributing to viscosity, the inventors have surprisingly found that the viscosity drop in mesalazine suspensions that is observed in the presence of isotonicity agent can be more than compensated by using disintegrating agents in quantities in excess of what is required to obtain a homogeneous suspension. The disintegrating agents that can be used in the granules of the invention include, but are not limited to, crospovidone, croscarmellose sodium, sodium starch glycollate and polyacrillin potassium. In one embodiment, the disintegrating agent comprised in the mesalazine granules is selected from the group consisting of crospovidone, croscarmellose sodium, sodium starch glycollate and polyacrillin potassium. The disintegrating agent preferably is present in an amount from 10% to 30% w/w, preferably from 15 to 25% w/w, based on the total weight of the granules.

The inventors have found that suspensions having a preferred combination of isotonicity, viscosity and homogeneity are obtainable from granules of mesalazine wherein the isotonicity agent and the disintegrating agent are present in a ratio by weight ranging from 1:0.8 to 1:2.5.

Hence in yet another aspect of the present invention, granules are provided comprising mesalazine as an active ingredient, an isotonicity agent and a disintegrating agent. In one particular embodiment, in the granules according to the invention, the isotonicity agent and the disintegrating agent are present in a ratio by weight ranging from 1:0.8 to 1:2.5. Preferably, the isotonicity agent and the disintegrating agent are present in a ratio by weight ranging from 1:0.8 to 1:1.5. More preferably, the isotonicity agent and the disintegrating agent are present in a ratio by weight ranging from 1:0.9 to 1:1.2.

In yet another embodiment, in the granules according to the invention the disintegrating agent and the viscosity agent are present in a ratio by weight ranging from of 1:0.1 to 1:0.5. In a preferred embodiment, the disintegrating agent and the viscosity agent are present in a ratio by weight ranging from of 1:0.2 to 1:0.5. The granules of the invention are capable of providing a stable, homogeneous and isotonic suspension.

Even though the granules of the invention can contain additional therapeutically active ingredients that can contribute functionally to the main active ingredient mesalazine, the granules of the invention can be put to desired therapeutic use even when the granules contain mesalazine as the sole active ingredient. Preferably, the pharmaceutical composition or the kit of parts or the dry enema preparation of the invention contains mesalazine in an amount ranging from 500 to 5000 mg. More preferably, the pharmaceutical composition or the kit of parts or the dry enema preparation of the invention contains mesalazine in an amount ranging from 1000 to 4000 mg.

In one embodiment, the pharmaceutical composition or the kit of parts or the dry enema preparation of the invention comprises mesalazine granules comprising from 30% to 70% w/w of mesalazine, preferably the amount of mesalazine ranges from 34% to 65% w/w; and from 5% to 15% w/w of a viscosity agent, preferably the viscosity agent ranges from 7% to 13% w/w; and from 10% to 30% w/w of a disintegrating agent, preferably the disintegrant ranges from 15 to 25% w/w; and from 10% to 30% w/w of an isotonicity agent, preferably the isotonicity agent ranges from 13 to 28% w/w. Said w/w percentages are percentages based on the total weight of the granulate.

Suitably, additional pharmaceutical excipients may be present in the pharmaceutical composition or the kit of parts or the dry enema preparation of the invention, such as chelating agents, buffering agents and/or antioxidants Said excipients may be present both intragranular and/or extragranular.

The mesalazine granulate of the present invention may be prepared by techniques commonly known in the art. Suitably, the granulate may be prepared using wet granulation. Wet granulation may be carried out using fluid bed granulator or high shear mixer. After granulation, the obtained granulate may be dried and sized.

The mesalazine granules of the present invention may be tableted or encapsulated or packaged in a single dose container such as a stick pack or a sachet.

A suitable enema bottle to be included in the pharmaceutical composition or the kit of parts of the invention is known the skilled person. The enema bottle may be any suitable bottle that is commercially available. Preferably, a harmonica type bottle is used. In the present invention, preferably the enema bottle is suitable for containing a volume between 50 to 150 ml. Preferably, the enema bottle can contain a volume of 100 ml.

The enema bottle comprises a one-way valve. One-way valves suitably prevent leakage, regulate the flow of the suspension and/or keep the bottle collapsed after medication has been applied. One-way valves are state of the art and can be adapted to suit the specific conditions of the suspension as formed, such as for example the viscosity of the suspension.

In a preferred embodiment of the present invention, the mesalazine granulate is supplied in a package for at least one dosage together with the enema bottle and together with manual instructions about how to prepare and use the enema.

In one embodiment the invention provides granules wherein the granules when reconstituted with water to a volume of 100 ml at 25° C. form a suspension having a viscosity greater than 200 cps and less than 500 cps. In a preferred embodiment, the granules form a suspension having a viscosity ranging from 220 cps to 480 cps. Beyond a viscosity of 500 cps, the mesalazine suspension is difficult to be administered rectally. Below a viscosity of 200 cps, the mesalazine suspension tends to be unstable and show signs of particles settling down.

In one embodiment the granules of the invention are free from a preservative. In one embodiment, the pharmaceutical composition or the kit of parts or the dry enema preparation of the invention is free from a preservative.

The invention is further illustrated by way of the following non limiting examples. In the experiments displayed in table 1, viscosity of the suspensions was measured using a Brookfield's viscometer equipped with spindle 3 operated at 50 rpm.

### EXAMPLES

### Example 1

### Preparation of Granules

In each experiment displayed in table 1 below, the active ingredient is mixed with the excipients in their respective amounts and weight ratios as displayed in the table in a rotor mixer granulator (RMG) and granulated using water as the binder. The granules were then dried in a dryer for 60 minutes at 60° C. The dried granules were then passed through a 1.0 mm mesh screen.

### Example 2

### Preparation of Suspension

3.95 g of granules, prepared as in example 1, containing 2 g of mesalazine was reconstituted to 100 ml with water (25° C.) in a container by manually shaking twice for a period of 30 seconds each separated by a standing period of 5 minutes. The viscosity of the suspension was measured using a Brookfield's viscometer equipped with spindle 3 operated at 50 rpm.

TABLE 1
Comparative study of mesalazine formulation under varied relative
amounts of excipients

| | | | | | ratio isotonic | ratio | viscosity of |
|---|---|---|---|---|---|---|---|
| Exp | Mesalazine | Disintegrant | Viscosity | isotonic | agent to | disintegrant to | suspension |
| No. | (mg) | (mg) | agent (mg) | agent (mg) | disintegrant | viscosity agent | (cps) |
| 1 | 4000 | 600 | 300 | 0 | 0 | 1:0.5 | 340 |
| 2 | 4000 | 600 | 300 | 800 | 1:0.75 | 1:0.5 | 200 |
| 3 | 4000 | 600 | 600 | 800 | 1:0.75 | 1:1 | 600 |
| 4 | 2000 | 560 | 450 | 825 | 1:0.68 | 1:0.80 | No uniform |
| | | | | | | | suspension |
| | | | | | | | formed |
| 5 | 2000 | 750 | 375 | 825 | 1:0.91 | 1:0.5 | 220 |
| 6 | 2000 | 900 | 375 | 825 | 1:1 | 1:0.4 | 380 |
| 7 | 2000 | 2062.5 | 375 | 825 | 1:2.5 | 1:0.2 | 480 |
| 8 | 1000 | 390 | 450 | 825 | 1:0.47 | 1:1.5 | No uniform |
| | | | | | | | suspension |
| | | | | | | | formed |

From table 1, it is clear that suspensions having a desired viscosity, greater than 200 cps but less than 500 cps, are obtained, see experiments 5, 6 and 7, from granules in which the isotonicity agent and the disintegrating agent are present in a ratio by weight ranging from above 1:0.75 to 1:2.5. When the amount of disintegrating agent relative to the amount of isotonicity agent is less, see experiments 4 and 8, the granules failed to disintegrate resulting in formation of no uniform suspension. When the amount of disintegrating agent was progressively increased, from 750 mg to 20162 mg, without affecting any change in the amount of viscosity agent, surprisingly, there was concomitant increase in viscosity, see experiments 4, 5 and 6. Therefore, it is clear from the experiments displayed in table 1 that suspensions with viscosity in the desired range could be obtained by using excess amounts of disintegrating agent instead of increasing the quantity of viscosity agent. This is advantageous since desired viscosity is achieved—in contrast to the case where agents are used to increase the viscosity—in granules that use an isotonicity agent, without affecting the disintegration of said granules.

The present invention provides granules that can readily disintegrate to form suspensions that are isotonic with blood plasma as well as possess adequate viscosity and homogeneity required for a stable suspension suitable as retention enema. By enabling increase in the viscosity of suspensions by adding disintegrating agents in excess of what is required for formation of a particle free suspension, granules that can result in suspensions having a combination of desired isotonicity, homogeneity and viscosity are obtained. This remarkably eases the commercial manufacture of mesalazine granules intended to be used as suspensions for enema. Apart from the convenience of transport, the granules of the invention carry with them mesalazine along with the excipients in relative amounts suitable to form homogeneous, isotonic and stable suspensions suitable to be rectally administered. Further, the granules of the invention enable preparation of mesalazine suspensions free from any preservative.

The above description is illustrative only and is not limiting. The present invention is defined by the claims that follow and their full range of equivalents.

1-12. (canceled)

13. A pharmaceutical composition comprising:
a. mesalazine granules suitable for reconstitution into an enema and
b. an enema bottle comprising a one-way valve.

14. The pharmaceutical composition according to claim 13, wherein the enema bottle is bio-degradable.

15. The pharmaceutical composition according to claim 13, wherein the mesalazine granules comprise an isotonicity agent, a viscosity agent and/or a disintegrating agent.

16. The pharmaceutical composition according to claim 15, wherein the isotonicity agent and the disintegrating agent are present in a ratio by weight ranging from 1:0.8 to 1:2.5.

17. The pharmaceutical composition according to claim 15, wherein the disintegrating agent and the viscosity agent are present in a ratio by weight ranging from 1:0.1 to 1:0.5.

18. The pharmaceutical composition according to claim 13, wherein the mesalazine granules comprise, based on the total weight of the granules:
a. from 30% to 70% w/w of mesalazine; and
b. from 5% to 15% w/w of a viscosity agent;
c. from 10% to 30% w/w of a disintegrating agent; and
d. from 10% to 30% w/w of an isotonicity agent.

19. The pharmaceutical composition according to claim 15, wherein the isotonicity agent is sodium chloride.

20. The pharmaceutical composition according to claim 15, wherein the viscosity agent is a gum.

21. The pharmaceutical composition according to claim 15, wherein the disintegrating agent is croscarmellose sodium.

22. The pharmaceutical composition according to claim 13, wherein the mesalazine granules when reconstituted with water to a volume of 100 ml at 25° C. forms a suspension having a viscosity greater than 200 cps and less than 500 cps.

23. The pharmaceutical composition according to claim 13, comprising mesalazine or its pharmaceutically acceptable salt in an amount from 500 to 5000 mg.

24. The pharmaceutical composition according to claim 13, wherein the mesalazine granules are present in a monodose container.

25. The pharmaceutical composition according to claim 15, wherein the isotonicity agent and the disintegrating agent are present in a ratio by weight ranging from 1:0.8 to 1:1.5.

26. The pharmaceutical composition according to claim 15, wherein the disintegrating agent and the viscosity agent are present in a ratio by weight ranging from 1:0.2 to 1:0.5.

27. The pharmaceutical composition according to claim 14, wherein the mesalazine granules comprise, based on the total weight of the granules:
a. from 30% to 70% w/w of mesalazine;
b. from 5% to 15% w/w of a viscosity agent;
c. from 10% to 30% w/w of a disintegrating agent; and
d. from 10% to 30% w/w of an isotonicity agent.

28. The pharmaceutical composition according to claim 18, wherein the mesalazine granules comprise, based on the total weight of the granules:
a. from 34% to 65% w/w of mesalazine; and
b. from 7% to 13% w/w of a viscosity agent;
c. from 15% to 25% w/w of a disintegrating agent; and
d. from 13% to 28% w/w of an isotonicity agent.

29. The pharmaceutical composition according to claim 16, wherein the isotonicity agent is sodium chloride.

30. The pharmaceutical composition according to claim 16, wherein the viscosity agent is a gum.

31. The pharmaceutical composition according to claim 16, wherein the disintegrating agent is croscarmellose sodium.
